(19) United States
(12) Patent Application Publication
(10) Pub. No.: US 2023/0381073 A1
(43) Pub. Date: Nov. 30, 2023

(54) SKIN TREATMENT COMPOSITIONS AND METHODS

(71) Applicant: Paper Route Girls LLC, Newport Beach, CA (US)

(72) Inventors: Nancy Pellegrino, Villa Park, CA (US); John Garruto, Encinitas, CA (US); Bethany McCarver, Oceanside, CA (US)

(21) Appl. No.: 18/033,186

(22) PCT Filed: Oct. 21, 2021
(86) PCT No.: PCT/US2021/055976

§ 371 Date: Apr. 21, 2023

Related U.S. Application Data
(60) Provisional application No. 63/104,913, filed on Oct. 23, 2020.

(51) Int. Cl. A61K 8/365; A61Q 19/10

(52) U.S. Cl. CPC A61K 8/365; A61Q 19/10; A61K 2800/28; A61K 2800/30; A61K 2800/594

(57) ABSTRACT

Skin treatment compositions and methods provide a superficial depth skin peel and exfoliation utilizing a combination of four acids, a carrier gel, colorant(s), and a topical oxygen delivery system. The combination of acids, oxygen delivery, gel formulation and colorant(s) aid in the efficacy of the formulations to result in beneficial skin treatment compositions by creating an effective superficial peel without the use of BHA's and only using AHA's having a molecular weight greater than 100. The AHA's used in the present invention are intended to predominately impact the stratum corneum, the outermost layer of skin. The AHA's utilized in the present invention reduce corneocyte adhesion by impacting the ionic bonds in a manner that reduces the cohesion forces. The corneocytes are then more easily shed and replaced by the newer, bright layers underneath. This can give the skin a smoother and less scaly appearance.

### FIELD OF THE INVENTION

The compositions and methods disclosed herein relate to the treatment of human skin for improving the appearance thereof.

### BACKGROUND OF THE INVENTION

There are numerous skincare products and procedures used for improving the appearance of human skin. Some of the procedures and the products used in the procedures are performed only by a medical professional. Other products are designed for use by the consumer without the supervision or need for a medical professional.

Some of the more effective products designed for use by consumers employ topically applied compositions containing one or more active ingredients known to beneficially affect the skin. For example, compositions containing retinoids, particularly retinol, have proven to be effective in combatting fine lines, wrinkles and other indications of skin aging such as sagging. Topically applied retinoids promote the formation of collagen and elastin in the skin.

Many medical professional employ chemical peels to rejuvenate and improve the appearance of a patient's skin. In fact, chemical peels performed by a medical professional are the third most commonly performed noninvasive cosmetic procedure in the United States. The goal of a chemical peel is to remove layers of damaged skin and induce skin rejuvenation to occur, while at the same time minimizing complications, such as scarring and unwanted pigmentary change.

Chemical peels are typically divided into several categories, based upon the depth of penetration into the skin by the chemical agent used in the procedure. One such division, proposed by Mark G. Rubin, MD in the Manual of Chemical Peels Superficial and Medium Dept has four categories i.e. very superficial, superficial, medium, and deep. Rubin describes a very superficial peel as a peel that thins or removes the stratum corneum and does not create a wound below the stratum granulosum, a superficial peel as a peel that creates necrosis of part of all of the epidermis, a medium peel as a peel that creates necrosis of the epidermis and part or all of the papillary dermis, and a deep peel as a peel the creates necrosis of the epidermis and papillary dermis.

Chemical peeling is basically an accelerated form of exfoliation (a process that removes dry, dead skin cells from the surface of the skin) induced by the use of a chemical agent, typically containing one or more alpha hydroxy acids (AHA) or one or more beta hydroxy acids (BHA) or a combination of AHA's and BHA's. Chemical peeling creates changes in the skin by removing certain layers of damaged skin, wounding or destroying skin to stimulate epidermal growth, and inducing the production of new collagen resulting in new tissue or skin being constructed The deeper the level of the peel the more necrosis of the epidermis takes place; thereby increasing the amount of irritation to the skin and the length of recovery.

Generally, the lower the pH of the acid used in a chemical peel agent the stronger the agent and the deeper it will penetrate into the skin. In addition, BHA's due to their solubility in oil have an enhanced ability to penetrate the skin deeper than AHA's, which are water soluble. The most commonly used AHA in a chemical peel agent is glycolic acid (“There's a reason glycolic is the acid used the most in anti-aging products and doctor's-office peels,” “It does the job.” Ranella Hirsch, former president of the American Society of Cosmetic Dermatology and Aesthetic Surgery; The most popular and time-tested superficial peeling agent is glycolic acid (A Practical Approach to Chemical Peels A Review of Fundamentals and Step-by-step Algorithmic Protocol for Treatment, J Clin Aesthet Dermatol. 2018 August; 11(8): 21-28)). Glycolic acid is typically available in at-home treatments in 8 to 15 percent concentrations. Its lower molecular weight allows the glycolic acid to penetrate the stratum corneum more easily than higher molecular weight AHA's such as malic acid and mandelic acid. The most commonly used BHA's is salicylic acid.

In addition to pH, the percentage of free acids in the chemical agent will impact the depth of a peel. Free acid refers to the amount or concentration of acid actually available. Generally, the higher the concentration of free acid in the chemical peeling agent, the deeper and larger the injury caused to the skin. For example, a 70% glycolic acid peel will be a more aggressive peel than a 30% glycolic acid peel when used on the same patient.

Chemical peeling agents using glycolic and BHA acids typically require a neutralizer to terminate their action after the desired result is achieved or a specified time period has expired, otherwise the acids may penetrate too deeply and damage the skin more significantly than was intended. Neutralization is typically accomplished by applying an alkaline pH or diluting the chemical peeling agent by flushing the treated area with water. Neutralization presents certain difficulties. For example, applying water to dilute the acid does not immediately terminate the effect of the acid and may not remove all of the chemical peeling agent, which could result in additional unwanted peeling by the acid. In the instances in which a neutralizing agent is used, it may be difficult to determine whether the acid has been neutralized. A neutralizing agent such as sodium bicarbonate will cause bubbling on the surface of the skin to indicate the acid is being neutralized but the sodium bicarbonate must be applied in generous amounts to the treated area resulting in dripping of the solution from the treated area; thereby creating a mess.

Chemical peels administered by a medical professional typically have a pH and acid concentration that is lower than the pH and acid concentration of chemical peel products that are considered safe for use by consumers. For example, some professional grade chemical peels have a pH level between 1.0 and 2.5, with an acid concentration range of 35-70%. A typical consumer product will have a higher pH level. The Cosmetic Ingredient Review Expert Panel recommends that chemical peels employing AHA's when used in products intended for the retail market and have concentrations of no greater than 10% by weight of the composition at pHs no less than 3.5.

Many of the consumer products purporting to create a superficial chemical peel use a combination of AHA's and BHA's. For example, the ingredients of the product T.L.C. Sukari Babyfacial sold by Drunk Elephant include a 25% by weight AHA blend of glycolic, tartaric, lactic, and citric acids with 2% BHA and Alpha Beta® Extra Strength Daily Peel sold by Dr. Dennis Gross has a combination of glycolic acid (AHA), salicylic acid (BHA) and lactic acid (AHA).

Other known chemical peel products that are commercially available are described below:

### The Microdelivery Resurfacing Peel (Requires Two Steps)

Step one—Vitamin C/Peptide Resurfacing Crystals: Apply a teaspoon amount to clean, dry skin. Once applied, crystals should appear almost like a mask. Gently massage in a circular motion over entire face for up to 60 seconds, avoiding eye area. Do not scrub; use less pressure for sensitive skin.

Step two—Lactic/Salicylic Acid Activating Gel: Place a generous teaspoon amount in the palm of your hand. Massage gel onto fingertips and apply on top of crystals. As activator is applied, a warming sensation will occur. White foam will appear, indicating activation. Leave foam on for two to three minutes to infuse vitamin C and peptides into the skin. You can massage foam for extra exfoliating benefits or let it sit for sensitive skin. Rinse thoroughly. You can use a washcloth for removal to avoid getting the peel in your hair.

Dr. Dennis Gross Skincare Alpha Beta® Universal Daily Peel (Two-Step Process)

A patented two-step anti-aging AHA/BHA peel pad pre-dosed with five acids to visibly reduce dullness and uneven texture, pores, and dark spots, now also available in a 20th Anniversary limited-edition 35-pack reusable tin.

### Lancer Skincare Caviar Lime Acid Peel (Uses Glycolic Acid)

Caviar Lime Extract: Promotes skin exfoliation.
Glycolic Acid 10% by weight: Removes dry, dulling cells, improving the look of pores and skin texture.
Phytic Acid 10% by weight: Brightens and evens complexion.
Papaya and Pineapple Enzymes: Smooth and soften skin.
   Dr. Dennis Gross Skincare

### Alpha Beta Extra Strength Daily Peel (has Retinol)

Ferulic Acid: Enhances properties of other vitamins for healthy skin and offers antioxidant protection. —Retinol: Helps smooth wrinkles and lines while increasing moisture and the appearance of elasticity. —Bisabolol (derived from chamomile): Soothes.
Rehmannia glutinosa Root Extract: Supports natural collagen and acid efficiency.

### Kate Somerville RetAsphere™ Micro Peel (Uses Retinol and Glycolic Acid)

Retasphere Smart Release™ Carrier System: Encapsulates pure Retinol in a lipid shell so it can penetrate deep into the epidermis, where it is most effective, to help diminish the look of fine lines and wrinkles; utilizes time-release absorption and melts gently into the skin to help prevent irritation and dryness.
10% by weight Glycolic Acid: Derived from sugar cane and beets, it gently exfoliates the uppermost layer of skin to reveal a fresh, renewed complexion instantly.
Natural Lactose: Helps skin renew at an optimal rate to help prevent dryness and flaking
Hyaluronic Acid: Delivers a surge of moisture to visibly plump and smooth the skin's surface.

The following patents relate to various skin treatment compositions and methods: U.S. Pat. No. 9,782,334 Systems and Methods for Skin Rejuvenation; U.S. Pat. No. 9,241,887 Systems and Methods for Skin Rejuvenation; U.S. Pat. No. 8,568,749 Systems and Methods for Skin Rejuvenation; U.S. Pat. No. 8,449,923 Methods for Inhibiting Sensory Nerves by Topically Administering Strontium Containing Compositions to Keratinized Skin; U.S. Pat. No. 8,147,855 Methods for Inhibiting Sensory Responses in the Skin such as Pain and Itch Using Topical Product Formulations Containing Strontium; U.S. Pat. No. 7,723,387 Self-Neutralizing Acid Peel for Dermatologic Use; U.S. Pat. No. 7,404,967 Topical Product Formulations Containing Strontium for Reducing Skin Irritation; U.S. Pat. No. 5,505,948 Topical Product Formulations Containing Strontium for Reducing Skin Irritation; U.S. Pat. No. 7,189,406 Home Skin Peel Composition for Producing Healthy and Attractive Skin; U.S. Pat. No. 8,017,138 Composition and Method for Treating Skin; and U.S. Pat. No. 5,720,963 Barrier Disruption Treatments for Structurally Deteriorated Skin.

### SUMMARY OF THE INVENTION

The present invention is intended to create an effective superficial peel without the use of BHA's and only using AHA's having a molecular weight greater than 100. The AHA's used in the present invention are intended to predominately impact the stratum corneum, the outermost layer of skin. The stratum corneum is comprised of rigid corneocytes (keratin-filled dead cells) in a specialized lipid matrix. Corneocytes are held or adhere together by ionic bonding. The AHA's utilized in the present invention reduce corneocyte adhesion by impacting the ionic bonds in a manner that reduces the cohesion forces (A theory for the mechanism of action of the alpha-hydroxy acids applied to the skin Med Hypotheses. 1999 November; 53(5):380-2.) The corneocytes are then more easily shed and replaced by the newer, brighter layers underneath. This can give the skin a smoother and less scaly appearance.

In general terms, the skin treatment compositions and methods disclosed herein provide a superficial depth skin peel and exfoliation utilizing a combination of four acids, a carrier gel, colorant(s), and a topical oxygen delivery system. The combination of acids, oxygen delivery, gel formulation and colorant(s) aid in the efficacy of the formulations to result in beneficial skin treatment compositions.

### DETAILED DESCRIPTION OF THE INVENTION

One mode of the current invention uses a combination of three AHA's, i.e. mandelic acid, phytic acid and malic acid, with succinic acid, a dicarboxylic acid, to impact the stratum corneum. Each of the AHA's used is an exfoliating acid that produce results in a peel that are more subtle than that of a glycolic acid peels; however, the side effects and subsequent downtime of these acids are comparatively less, which allows for more frequent treatment sessions.

Mandelic is the largest AHA molecule (152.1473 g/mol), which means it penetrates the epidermis slowly, making it a good choice as an exfoliating agent for sensitive skin. It is soluble in both water and polar organic solutions, which results in a more uniform penetration through lipid-rich areas of skin. (Aston J G, Newkirk J D, Jenkins D M, Dorsky J. Mandelic acid. Org Synth. 1952; 3:538.) Malic acid is also a large molecule (134.0874 g/mol) when compared to other alpha hydroxy acids (AHA's), such as glycolic (76.05 g/mol) or lactic acid (90.08 g/mol), which decreases its penetration into the skin. In addition to being an exfoliant it can act as a humectant, something that attracts moisture to and traps it in the skin, and may have an antibacterial function. Phytic acid (660.04 g/mol) functions more as an antioxidant than a true exfoliating agent, making it is less irritating to the skin. It has a slow release, which promotes continuous penetration of the skin, and requires no external neutralization. Succinic acid (118.09 g/mol) is known to provide antimicrobial activity, increase the production of collagen and elastin, and enhance mitochondrial activity in skin cells and thus has use in products claiming skin energizing and revitalizing effect.

A person of ordinary skill in the art will appreciate that other AHA's, and dermatologically effective derivatives of AHA's, may also exhibit similar functionality when used in concentrated solutions in lieu of Mandelic acid, Phytic acid, Succinic acid and Malic acid, and such functional equivalents of AHA's are intended to be within the spirit and scope of the presently disclosed embodiments.

Medical professionals when performing a chemical peel will monitor the patient's skin and neutralize the acids if the acids are perceived as progressing too deeply. The present invention provides a composition and a method for at-home chemical skin peeling which is effective at causing exfoliation but gentle due to the active skin peeling ingredients not including any BHA's or AHA's having a molecular weight lower than 100 g/mol and having concentrations of the AHA's no greater than 10%, which is far lower than that utilized in dermatologists' and aestheticians' offices. Furthermore, the present invention does not need to be removed in a specified period of time or neutralized because the acids used are self-neutralizing.

To facilitate the work done by the chemical peeling agents of the present invention, the invention incorporates oxygenated perfluorocarbons. Oxygen doesn't dissolve as well in water, so instead it is dissolved in chemicals called perfluorocarbons, which are stable, unreactive carbon-based compounds containing fluorine atoms. They can dissolve 20 times more oxygen than water. In the current invention the thickness of the product itself is enough to hold the oxygen until it is massaged out after being applied on the skin.

Oxygenation of the skin has multiple benefits. It helps slow down the aging process, minimizes fine lines and wrinkles, rejuvenates the skin, lightens and brightens skin, fights and kills bacteria. It has been found that extra oxygen helps with wound healing (S Kellar, R G Audet, D F Roe, L A Rheins & Z D Draelos, Topically delivered dissolved oxygen reduces inflammation and positively influences structural proteins in healthy intact human skin, J Cosmet Dermatol 2013, 12, 86-95.) and lack of oxygen is said to encourage the proliferation of anaerobic bacteria such as p. acnes.

The present invention uses Gransil SIW-7100, available for purchase from Grant Industries, as the delivery system for its oxygenated perfluorocarbons. Gransil SIW-7100 gel entraps methyl perfluorobutyl esters (MPBE) into an elastomer matrix with an external water phase to manage evaporation in manufacturing and upon application. Once exposed to air, the oxygenated perfluorocarbons start to release the trapped oxygen in the form of bubbles. The bubbles are characterized by having negative electrical charges. The negative charge attracts dirt, debris and impurities as well as suspended floating particles very effectively. These bubbles help exfoliate dead skin cells and unclog pores while delivering the chemical peeling agents deeper into the skin. The oxygen delivery eliminates the need typically required by home chemical peel products to prep the skin with a alcohol, acetone or some other means.

The use of surfactants/emulsifying agents with chemical peeling agents has been recognized to improve the performance of the agents and allow for the use of lower concentrations of the acids comprising the chemical peeling agents to achieve the same result as higher concentrations without the use of a surfactant/emulsifying agent. Preferably the surfactants comprise about 5% to 25% by weight of the composition of the product. The surfactant used in the present invention is cocamidopropyl betaine. Other surfactants that are considered suitable for use in the present invention include are known to those of ordinary skill in the art.

Application of the currently available chemical peeling agents to the skin of a wearer has, however, many problems and operations of application. They tend to produce uncovered portions of the skin or result in uneven applications of the peeling agent. It is important for the user to know whether the product of the present invention has been applied to all the surfaces of the skin to be treated. To provide the consumer with that ability, the present invention includes synthetic fluorophlogopite in combination with titanium dioxide, iron oxides and tix oxide in a range of 1% to 5% by weight of the composition to give the product a gold color that is easily distinguishable from the surface of the skin; thereby allowing the user to easily determine the surfaces of the skin on which the product has been applied.

The pH of cosmetics and skin care products is important to maintain the skin's normal pH as closely as possible. As a result, many cosmetic products use sodium hydroxide in to establish and hold the pH of a product. In the present invention, sodium hydroxide is used in the range of 0.2 to 3% by weight of the composition.

The compositions of the invention results in beneficial effects due to the exfoliation caused by the acid combination; antioxidant functions; humectant functions; pH balancing; oxygenation of the skin through a bubbling delivery of oxygen to the skin by means of oxygenated perfluorocarbons; reduce-concentration acids due to the use of surfactants/emulsifying agents; a unique colorant system to visually aid in even and full skin coverage; and pH balancing to closely emulate the normal pH of human skin.

Table 1 below provides exemplary components and % by weight ranges for each component to achieve the beneficial results of the invention.

TABLE 1

| | | % Range |
|---|---|---|
| | | By |
| Trade Name | INCL Name | Weight |
| Chembetaine C | Cocamidopropyl Betaine | 5.0-25.0 |
| Chrono Chardy | Water, butylene glycol, | 0.01-2.0 |
| | vitis vinifera (grape) fruit | |
| | extract | |
| Deionized water | Water/aqua/eau | 30.0-50.0 |
| DL-Mandelic acid MA160 | Mandelic acid | 05.-2.0 |
| Euxyl PE 9010 | Phenoxyethanol, | 0.5-1.0 |
| | ethylhexylglycerin | |
| GRANSIL SIW-7100 | Methyl perfluorobutyl ether, | 10.0-30.0 |
| | isododecane, water | |
| Malic acid FCC | Malic acid | 05.-2.0 |
| Phytic acid extreme | Phytic acid | 0.5-2.0 |
| SEPIMAX ™ ZEN | Polyacrylate Crosspolymer-6 | 0.5-1.5 |
| Sepiplus 400 | Polyacrylate-13, polyisobutene, | 0.5-5.0 |
| | polysorbate 20 | |
| Sodium Hydroxide 50% | Water, sodium hydroxide | 0.2-3.0 |
| solution | | |
| Succinic acid, FCC S1686 | Succinic acid | 0.5-2.0 |
| Syncrystal spring gold | Synthetic fluorphlogopite, | 1.0-5.0 |
| | titanium dioxide (CI | |
| | 77891), iron oxides (CI | |
| | 77491), tin oxide | |

Set forth in Table 2 below is a further exemplary composition formulation in accordance with the principles of the invention.

TABLE 2

| P/N | Trade Name | INCI Name | % W/W |
|---|---|---|---|
| 1000 | Deionized water | Water/aqua/eau | 57.68 |
| 11184 | GRANSIL SIW-7100 | Methyl perfluorobutyl | 12.38 |
| | | ether | |
| 11184 | GRANSIL SIW-7100 | Isododecane | 9.5 |
| 11191 | Chembetaine C | Cocamidopropyl betaine | 3.6 |
| 11118 | Syncrystal Spring Gold | Synthetic fluorphlogopite | 2.46 |
| 6012 | Sepiplus 400 | Polyacrylate-13 | 1.8 |
| 11118 | Syncrystal Spring Gold | Titanium dioxide | 1.36 |
| 11184 | GRANSIL SIW-7100, | Butylene glycol | 1.3 |
| | Chrono Chardy | | |
| 11184 | GRANSIL SIW-7100 | Polysilicone-11 | 1.25 |
| ≥1% | | | |
| 11242 | Succinic acid, FCC S1686 | Succinic acid | 1.00 |
| 8043 | Phytic acid extreme | Phytic acid | .50 |
| 11243 | Malic acid FCC | Malic acid | 1.00 |
| 10098 | DL-Mandelic acid MA160 | Mandelic acid | 1.00 |
| 11180 | Chrono Chardy | Vitis Vinifera | 0.04 |
| | | (Grape (Fruit extract | |
| 11184 | GRANSIL SIW-7100 | Decyl glucoside | 0.05 |
| 10548 | SEPIMAX ™ ZEN | Polyacrylate | 0.80 |
| | | Crosspolymer-6 | |
| 11191 | Chembetaine C | Glycerin | 0.45 |
| 6012 | Sepiplus 400 | Polysorbate 20 | 0.15 |
| 4695 | Euxyl PE 9010 | Ethylhexylglycerin | 0.09 |
| 6012 | Sepiplus 400 | Sorbitan isostearate | 0.12 |
| 11191 | Chembetaine C | Sodium glycolate | 0.11 |
| 111184 | GRANSIL SIW-7100 | Hexylene glycol | 0.03 |
| 11184 | GRANSIL SIW-7100 | Caprylyl glycol | 0.03 |
| 6012 | Sepiplus 400 | Polyisobutene | 0.84 |
| 4695 | Euxyl PE 9010, | Phenoxyethanol | 0.79 |
| | GRANSIL SIW-7100 | | |
| 11191 | Chembetaine C | Sodium chloride | 0.90 |
| 9198 | Sodium Hydroxide 50% | Sodium hydroxide | 0.61 |
| | solution | | |
| 11118 | Syncrystal Spring Gold | Tin oxide | 0.02 |
| 11118 | Syncrystal Spring Gold | Iron oxides (CI 77491) | 0.16 |

Set forth below in Table 3 is an explanation of the various components in the composition in accordance with the invention explaining their function and a general description of each component.

TABLE 3

| P/N | Trade Name | INCI | Function | Description | Crossover |
|---|---|---|---|---|---|
| 10743 | Bio - Based Succinic | Disco succinic acid | Exfoliation/hyperpigmentation | Bio-based succinic acid is a | Peel |
| | Acid 99.5% | | | natural, plant-derived | |
| | | | | building block that can be | |
| | | | | used to formulate a broad | |
| | | | | range of bio-based esters for | |
| | | | | skin care and hair care, and | |
| | | | | as a direct ingredient with | |
| | | | | properties for effervescence | |
| | | | | and exfoliation | |
| 9443 | Malic acid FCC USP | Malic acid | Exfoliation/hyperpigmentation | Malic acid improves | Peel |
| | Powder | | | hyperpigmentation by | |
| | | | | decreasing the production of | |
| | | | | melanin | |
| 10098 | DL-Mandelic acid | Mandelic acid | Exfoliation/Sebum production | Mandelic acid is extractd | Peel |
| | MA160 | | | from the bitter almon and ia | |
| | | | | sn alpha hydroxyl acid | |
| | | | | (AHA). Alpha hydroxy | |
| | | | | acids speed up the cellular | |
| | | | | turnover rate, ridding the | |
| | | | | skin of dead skin cells that | |
| | | | | enhance the natural creases | |
| | | | | in skin and dull the | |
| | | | | complexion | |
| 8043 | Phytic acid extreme | Phytic acid | Exfoliation/Free radical | Phytic acid extreme is a | Peel |
| | | | scavenging | naturally derived ingredient | |
| | | | | sourced from rice. It has a | |
| | | | | unique structure which | |
| | | | | provides self-neutralizing | |
| | | | | activity that yields gentle | |
| | | | | keratolytic effects and aids in | |
| | | | | cell turnover | |
| 11184 | GRANSIL SIW-7100 | Methyl perfluorobutyl | Fizzing/oxygenation | Topical oxygen delivery | Peel |
| | | ether, isododecane, | | system. O2 is a basic | |
| | | water, polysilicone-11, | | requirement in aerobic | |
| | | butylene glycol, decyl | | respiration in order to | |
| | | glucoside, caprylyl | | produce ATP cellular energy. | |
| | | glycol | | Anaerobic bacteria such as p. | |
| | | | | acnes proliferate in skin with | |
| | | | | under oxygenated conditions | |
| | | | | such as clogged pores. | |
| | | | | Wound healing becomes | |
| | | | | difficult in under-oxygenated | |
| | | | | skin. Diabetic and severely | |
| | | | | compromised skin conditions | |
| | | | | benefit from oxygen therapy | |
| 11180 | Chrono Chardy | Water, butylene glycol, | Circadian, brown spots from | Enhanced clock gene | Day, night, |
| | | Vitis Vinifera (Grape) | chardonnay | expression (circadian rhythm | peel |
| | | Fruit Extract | | of skin functions). | |
| | | | | Promoting nocturnal lipid | |
| | | | | transportation. Promoting | |
| | | | | diurnal filaggrin production. | |
| | | | | Improved barrier function, | |
| | | | | Moisturizing. Brown spots | |
| | | | | whitening. | |
| 11118 | Syncrystal Spring Gold | Synthetic | Effect pigment | Effect pigment | Peel, GR |
| | | fluorphlogopite, | | | |
| | | titanium dioxide | | | |
| | | (CI77891), iron oxides | | | |
| | | (CI 774911), tin oxide | | | |
| 1000 | Deionized water | Water/aqua/eau | | SOLVENT | |
| 4695 | Euxyl PE9010 | Phenoxyethanol, | | Preservative | |
| | | ethylhexylglycerin | | | |
| 9198 | Sodium hydroxide | Water, sodium | | pH adjuster | |
| | 50% solution | hydroxide | | | |
| 10548 | SEPIMAS ™ ZEN | Polyacrylate | | SEPIMAX ™ ZEN is an | |
| | | Crosspolymer-6 | | associative polymer with | |
| | | | | excellent resistance to | |
| | | | | electrolytes. It can produce | |
| | | | | clear aqueous gels with a rich | |
| | | | | and elegant touch lending to | |
| | | | | a velvety sensation | |
| 6012 | Sepiplus 400 | Polyacrylate-13, | | Multifunctional polymer with | |
| | | polyisobutene, | | silicone feel and glide-on | |
| | | polysorbate 20 | | spreading. Its optimal in pH | |
| | | | | range 3-12 and it tends to a | |
| | | | | greater thickening capacity in | |
| | | | | presence of electrolytes. | |
| | | | | This product is an | |
| | | | | emulsifying-stabilizing | |
| | | | | power of oil phases and | |
| | | | | compatible with solvents. It | |
| | | | | is also concentrated and per- | |
| | | | | neutralized as well as ready | |
| | | | | to use so it is ideal for cold | |
| | | | | process during introduction | |
| | | | | to oil or water phase | |
| 11191 | Chembetaine C | Cocamidopropyl | | Chembetaine ™ C surfactant | |
| | | betaine | | is a high-foaming, mild | |
| | | | | surfactant that is stable over | |
| | | | | a wide pH range. Its | |
| | | | | excellent foam and viscosity | |
| | | | | building with anionics | |
| | | | | suggest uses in bubble baths, | |
| | | | | liquid soaps, skin cleansers, | |
| | | | | shampoos and conditioners. | |

Set forth in Table 4 below are the compositions in an exemplary embodiment of the present invention and categorized into Parts A-D.

TABLE 4

| | Trade Name | INCL Name | Supplier/Distributor | % W/W | Weight | |
|---|---|---|---|---|---|---|
| Part A | Deionized water | Water/aqua/eau | Deionized water | 45.13 | 135.390 | g |
| | Succinic acid, FCC | Succinic acid | Spectrum Chemical | 1.00 | 3.000 | g |
| | S1686 | | | | | |
| | Malic acid FCC | Malic acid | Spectrum Chemical | 1.00 | 3.000 | g |
| | DL-Mandelic acid | Mandelic acid | Spectrum Chemical | 1.00 | 3..000 | g |
| | MA160 | | | | | |
| | Phytic acid extreme | Phytic acid | Biosil/Integrity | 1.00 | 3.000 | g |
| | | | Ingredients | | | |
| | Euxyl PE9010 | Phenoxyethanol, ethylhexylglycerin | Schulke/Ross | 0.85 | 2.550 | g |
| | | | Organic Specialty | | | |
| | | | Sale | | | |
| Part B | Sodium hydroxide 50% | Water, sodium hydroxide | Spectrum Chemical | 1.22 | 3.660 | g |
| | solution | | | | | |
| | SEPIMAX ™ ZEN | Polyacrylate crosspolymer-6 | Seppic, Inc. | 0.80 | 2.400 | g |
| Part C | Sepiplus 400 | Polyacrylate-13, Polyisobutene, Polysorbate 20 | Seppic, Inc. | 3.00 | 9.000 | g |
| | GRANSIL SIW-7100 | Methyl perfluorobutyl ether, Isododecane, water, | Grant Industries | 25.00 | 75.000 | g |
| | | polysilicone-11, butylene glycol, decyl | | | | |
| | | glucoside, caprylyl glycol | | | | |
| | Chrono Chardy | Water, butylene glycol, Vitis Vinifera (Grape) | Miyoshi | 1.00 | 3.000 | g |
| | | Fruit extract | America/Ichimaru | | | |
| | | | Pharcos | | | |
| | Syncrystal spring gold | Synthetic fluorphlogopite, titanium dioxide (CI | Eckart/EES Cosmetic | 4.00 | 12.000 | g |
| | | 77891), iron oxides (CI 77491), tin oxide | Solutions | | | |
| Part D | Chembetaine C | Cocamidopropyl betaine | Lubrizol/Coast | 15.00 | 45.000 | g |
| | | | Southwest | | | |

Set forth in Table 5 below is an exemplary method for preparing a composition in accordance with the invention.

TABLE 5
PROCEDURE:

In sanitized mixing vessel, add water in Part A. Add remaining ingredients in order shown ensuring
each ingredient is dissolved before the addition of the next. Adjust pH to 3.65-3.75 with Sodium
Hydroxide 50%. Add Sepimax Zen and mix until completely smooth and uniform avoiding aeration.
Add ingredients in Part C in order shown insuring each ingredient is completely mixed in before the
addition of the next. Add Cocamidopropyl Betaine from Part D and mix until uniform. Fill
immediately. DO NOT let bath sti in mixing vessel. This formula is VERY oxygen reactive.
*Sepiplus 400 must be mixed in it's original container before being weighed.
Lab and polit batches shuld be made prior to large scale manufacturing to ensure a smooth transition
from laboratory to production.
Quality - Physical Specifications - tentative specification ranges; will be finalized upon completion
of accelerated stability and pilot batch.
APPEARANCE: Semi-viscous emulsion
COLOR: Gold
ODOR: characteristic of base
pH @ 25° C.: 3.5-4.0
VISCOSITY @ 25° C.″ RVT: Spindle 5, 20 RPM: 7,000-17,000
SPECIFIC GRAVITY g/ml @ 25° C.: 1.01-1.08
MICROBIOLOGY: <10 cfu/GRAM, No pathogens
OPTIONAL: Centrifuge for 30 minutes at 3,500 RPM

While exemplary components and by weight percentage ranges are provided, it will be understood by persons skilled in the art that variations in the components and the weight percentage of each may be modified while achieving the beneficial results of the invention.

What is claimed is:

1. A skin treatment composition, comprising:
at least one AHA;
at least one oxygenated perfluorocarbon;
at least one pH balancing component, and
at least one surfactant or emulsifying agent.

2. The skin treatment composition of claim 1, further comprising a colorant composition.

3. A skin treatment composition, comprising:

| | | % Range |
|---|---|---|
| | | By |
| Trade Name | INCL Name | Weight |
| Chembetaine C | Cocamidopropyl Betaine | 5.0-25.0 |
| Chrono Chardy | Water, butylene glycol, | 0.01-2.0 |
| | vitis vinifera (grape) | |
| | fruit extract | |
| Deionized water | Water/aqua/eau | 30.0-50.0 |
| DL-Mandelic acid MA160 | Mandelic acid | 05.-2.0 |
| Euxyl PE 9010 | Phenoxyethanol, | 0.5-1.0 |
| | ethylhexylglycerin | |
| GRANSIL SIW-7100 | Methyl perfluorobutyl | 10.0-30.0 |
| | ether, isododecane, water | |
| Malic acid FCC | Malic acid | 05.-2.0 |
| Phytic acid extreme | Phytic acid | 0.5-2.0 |
| SEPIMAX ™ ZEN | Polyacrylate Crosspolymer-6 | 0.5-1.5 |
| Sepiplus 400 | Polyacrylate-13, polyisobutene, | 0.5-5.0 |
| | polysorbate 20 | |
| Sodium Hydroxide 50% | Water, sodium hydroxide | 0.2-3.0 |
| solution | | |
| Succinic acid, FCC S1686 | Succinic acid | 0.5-2.0 |
| Syncrystal spring gold | Synthetic fluorphlogopite, | 1.0-5.0 |
| | titanium dioxide (CI | |
| | 77891), iron oxides (CI | |
| | 77491), tin oxide | |

4. A skin treatment composition, comprising:

| P/N | Trade Name | INCI Name | % W/W |
|---|---|---|---|
| 1000 | Deionized water | Water/aqua/eau | 57.68 |
| 11184 | GRANSIL SIW-7100 | Methyl perfluorobutyl | 12.38 |
| | | ether | |
| 11184 | GRANSIL SIW-7100 | Isododecane | 9.5 |
| 11191 | Chembetaine C | Cocamidopropyl betaine | 3.6 |
| 11118 | Syncrystal Spring Gold | Synthetic fluorphlogopite | 2.46 |
| 6012 | Sepiplus 400 | Polyacrylate-13 | 1.8 |
| 11118 | Syncrystal Spring Gold | Titanium dioxide | 1.36 |
| 11184 | GRANSIL SIW-7100, | Butylene glycol | 1.3 |
| | Chrono Chardy | | |
| 11184 | GRANSIL SIW-7100 | Polysilicone-11 | 1.25 |
| ≥1% | | | |
| 11242 | Succinic acid, FCC S1686 | Succinic acid | 1.00 |
| 8043 | Phytic acid extreme | Phytic acid | .50 |
| 11243 | Malic acid FCC | Malic acid | 1.00 |
| 10098 | DL-Mandelic acid MA160 | Mandelic acid | 1.00 |
| 11180 | Chrono Chardy | Vitis Vinifera | 0.04 |
| | | (Grape (Fruit extract | |
| 11184 | GRANSIL SIW-7100 | Decyl glucoside | 0.05 |
| 10548 | SEPIMAX ™ ZEN | Polyacrylate | 0.80 |
| | | Crosspolymer-6 | |
| 11191 | Chembetaine C | Glycerin | 0.45 |
| 6012 | Sepiplus 400 | Polysorbate 20 | 0.15 |
| 4695 | Euxyl PE 9010 | Ethylhexylglycerin | 0.09 |
| 6012 | Sepiplus 400 | Sorbitan isostearate | 0.12 |
| 11191 | Chembetaine C | Sodium glycolate | 0.11 |
| 111184 | GRANSIL SIW-7100 | Hexylene glycol | 0.03 |
| 11184 | GRANSIL SIW-7100 | Caprylyl glycol | 0.03 |
| 6012 | Sepiplus 400 | Polyisobutene | 0.84 |
| 4695 | Euxyl PE 9010, | Phenoxyethanol | 0.79 |
| | GRANSIL SIW-7100 | | |
| 11191 | Chembetaine C | Sodium chloride | 0.90 |
| 9198 | Sodium Hydroxide 50% | Sodium hydroxide | 0.61 |
| | solution | | |
| 11118 | Syncrystal Spring Gold | Tin oxide | 0.02 |
| 11118 | Syncrystal Spring Gold | Iron oxides (CI 77491) | 0.16 |

5. A method of treating human skin, comprising;
applying to the skin a treatment composition comprising:
at least one AHA;
at least one oxygenated perfluorocarbon;
at least one pH balancing composition; and
at least one surfactant or emulsifying agent.

6. A method of treating human skin, comprising:
applying to the skin a treatment composition comprising:

| | | % Range |
|---|---|---|
| | | By |
| Trade Name | INCL Name | Weight |
| Chembetaine C | Cocamidopropyl Betaine | 5.0-25.0 |
| Chrono Chardy | Water, butylene glycol, | 0.01-2.0 |
| | vitis vinifera (grape) fruit extract | |
| Deionized water | Water/aqua/eau | 30.0-50.0 |
| DL-Mandelic acid MA160 | Mandelic acid | 05.-2.0 |
| Euxyl PE 9010 | Phenoxyethanol, | 0.5-1.0 |
| | ethylhexylglycerin | |
| GRANSIL SIW-7100 | Methyl perfluorobutyl ether, | 10.0-30.0 |
| | isododecane, water | |
| Malic acid FCC | Malic acid | 05.-2.0 |
| Phytic acid extreme | Phytic acid | 0.5-2.0 |
| SEPIMAX ™ ZEN | Polyacrylate Crosspolymer-6 | 0.5-1.5 |
| Sepiplus 400 | Polyacrylate-13, polyisobutene, | 0.5-5.0 |
| | polysorbate 20 | |
| Sodium Hydroxide 50% | Water, sodium hydroxide | 0.2-3.0 |
| solution | | |
| Succinic acid, FCC S1686 | Succinic acid | 0.5-2.0 |
| Syncrystal spring gold | Synthetic fluorphlogopite, | 1.0-5.0 |
| | titanium dioxide (CI | |
| | 77891), iron oxides (CI | |
| | 77491), tin oxide | |

7. A method of treating human skin, comprising:
applying to the skin a treatment composition comprising:

| P/N | Trade Name | INCI Name | % W/W |
|---|---|---|---|
| 1000 | Deionized water | Water/aqua/eau | 57.68 |
| 11184 | GRANSIL SIW-7100 | Methyl perfluorobutyl | 12.38 |
| | | ether | |
| 11184 | GRANSIL SIW-7100 | Isododecane | 9.5 |
| 11191 | Chembetaine C | Cocamidopropyl betaine | 3.6 |
| 11118 | Syncrystal Spring Gold | Synthetic fluorphlogopite | 2.46 |
| 6012 | Sepiplus 400 | Polyacrylate-13 | 1.8 |
| 11118 | Syncrystal Spring Gold | Titanium dioxide | 1.36 |
| 11184 | GRANSIL SIW-7100, | Butylene glycol | 1.3 |
| | Chrono Chardy | | |
| 11184 | GRANSIL SIW-7100 | Polysilicone-11 | 1.25 |
| ≥1% | | | |
| 11242 | Succinic acid, FCC S1686 | Succinic acid | 1.00 |
| 8043 | Phytic acid extreme | Phytic acid | .50 |
| 11243 | Malic acid FCC | Malic acid | 1.00 |
| 10098 | DL-Mandelic acid MA160 | Mandelic acid | 1.00 |
| 11180 | Chrono Chardy | Vitis Vinifera | 0.04 |
| | | (Grape (Fruit extract | |
| 11184 | GRANSIL SIW-7100 | Decyl glucoside | 0.05 |
| 10548 | SEPIMAX ™ ZEN | Polyacrylate | 0.80 |
| | | Crosspolymer-6 | |
| 11191 | Chembetaine C | Glycerin | 0.45 |
| 6012 | Sepiplus 400 | Polysorbate 20 | 0.15 |
| 4695 | Euxyl PE 9010 | Ethylhexylglycerin | 0.09 |
| 6012 | Sepiplus 400 | Sorbitan isostearate | 0.12 |
| 11191 | Chembetaine C | Sodium glycolate | 0.11 |
| 111184 | GRANSIL SIW-7100 | Hexylene glycol | 0.03 |
| 11184 | GRANSIL SIW-7100 | Caprylyl glycol | 0.03 |
| 6012 | Sepiplus 400 | Polyisobutene | 0.84 |
| 4695 | Euxyl PE 9010, | Phenoxyethanol | 0.79 |
| | GRANSIL SIW-7100 | | |
| 11191 | Chembetaine C | Sodium chloride | 0.90 |
| 9198 | Sodium Hydroxide 50% | Sodium hydroxide | 0.61 |
| | solution | | |
| 11118 | Syncrystal Spring Gold | Tin oxide | 0.02 |
| 11118 | Syncrystal Spring Gold | Iron oxides (CI 77491) | 0.16 |
